(19) United States
(12) Patent Application Publication
(10) Pub. No.: US 2026/0306292 A1
(43) Pub. Date: Oct. 8, 2026

(54) DEVICES, SYSTEMS AND METHODS FOR TREATING THE SKIN

(71) Applicant: HydraFacial LLC, Long Beach, CA (US)

(72) Inventors: Joseph Wong, South Pasadena, CA (US); Stacie Depner, Anaheim, CA (US); Aaron MacDonald, Squamish (CA)

(21) Appl. No.: 19/482,202

(22) PCT Filed: May 7, 2024
(86) PCT No.: PCT/US2024/028183

§ 371 Date: Nov. 6, 2025

Related U.S. Application Data
(60) Provisional application No. 63/464,867, filed on May 8, 2023.

(51) Int. Cl. A61F 7/00

(52) U.S. Cl. CPC A61F 7/007; A61F 2007/0052; A61F 2007/0056

(57) ABSTRACT

According to some embodiments, a handpiece comprises a main body, a tip positioned along a distal end of the main body, and a thermal conditioning assembly or module, the thermal conditioning assembly or module being configured to selectively heat and/or cool treatment fluid being transferred to the tip, wherein the thermal conditioning assembly or module comprises a fluid inlet and a fluid outlet, wherein a fluid pathway extending between the fluid inlet and the fluid outlet comprises at least one turn, wherein the thermal conditioning assembly or module comprising a heating or cooling component and at least one heat transfer component adjacent the heating or cooling component at or near the fluid pathway, and wherein the at least one heat transfer component is configured to selectively heat or cool treatment fluid passing through the fluid pathway when the heating or cooling component is electrically energized or activated.

### CROSS REFERENCE TO RELATED APPLICATIONS

This application claims the priority benefit of U.S. Provisional Patent Application No. 63/464,867, filed May 8, 2023, the contents of which are incorporated by reference herein and made a part of the present application in their entirety.

### BACKGROUND

### Field

This application relates generally to skin treatment, and more specifically, to apparatuses, systems and methods for treating a person's skin.

### Description of the Related Art

Abrasion of the outer layer or epidermis of the skin is desirable to smooth or blend scars, blemishes, or other skin conditions that may be caused by, for example, acne, sun exposure, aging and/or the like. Standard techniques used to abrade the skin have generally been referred to as dermabrasion and microdermabrasion. Such techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

### SUMMARY

According to some embodiments, a handpiece for use in skin treatment procedure comprises a main body, a tip positioned along a distal end of the main body, and a thermal conditioning assembly or module, the thermal conditioning assembly or module being configured to selectively heat and/or cool treatment fluid being transferred to the tip, wherein the thermal conditioning assembly or module comprises a fluid inlet and a fluid outlet, wherein a fluid pathway extending between the fluid inlet and the fluid outlet comprises at least one turn, wherein the thermal conditioning assembly or module comprising a heating or cooling component and at least one heat transfer component adjacent the heating or cooling component at or near the fluid pathway, and wherein the at least one heat transfer component is configured to selectively heat or cool treatment fluid passing through the fluid pathway when the heating or cooling component is electrically energized or activated.

According to some embodiments, a handpiece for use in skin treatment procedure comprises a main body, a tip positioned along a distal end of the main body, and a thermal conditioning assembly or module, the thermal conditioning assembly or module being configured to selectively heat and/or cool treatment fluid being transferred to the tip, wherein the thermal conditioning assembly or module comprises a fluid inlet and a fluid outlet, wherein a fluid pathway extending between the fluid inlet and the fluid outlet comprises at least one turn, wherein the thermal conditioning assembly or module comprises a heating or cooling component and at least one heat transfer component extending between the heating or cooling component at the fluid pathway, and wherein the at least one heat transfer component is configured to selectively heat or cool treatment fluid passing through the fluid pathway when the heating or cooling component is electrically energized.

According to some embodiments, treatment fluid is heated or cooled conductively. In some embodiments, treatment fluid is heated or cooled convectively.

According to some embodiments, the thermal conditioning assembly or module comprises an electric heater. In some embodiments, the thermal conditioning assembly or module comprises a thermoelectric device or another type of heat pump.

According to some embodiments, a method for treating skin comprises performing a skin treatment procedure using a tip of a handpiece, the handpiece comprising a main body and the tip, wherein the tip is positioned at or near a distal end of the main body, and selectively heating and/or cooling a treatment fluid being transferred to the tip using a thermal conditioning assembly or module of the handpiece, wherein selectively heating and/or cooling a treatment fluid comprises using a thermal conditioning assembly or module, wherein the thermal conditioning assembly or module comprises a fluid inlet and a fluid outlet, wherein a fluid pathway extending between the fluid inlet and the fluid outlet comprises at least one turn, wherein the thermal conditioning assembly or module comprising a heating or cooling component and at least one heat transfer component extending between the heating or cooling component at the fluid pathway, and wherein the at least one heat transfer component is configured to selectively heat or cool treatment fluid passing through the fluid pathway when the heating or cooling component is electrically energized.

According to some embodiments, treatment fluid is heated or cooled conductively. In some embodiments, treatment fluid is heated or cooled convectively. According to some embodiments, the thermal conditioning assembly or module comprises an electric heater. In some embodiments, the thermal conditioning assembly or module comprises a thermoelectric device or another type of heat pump.

According to some embodiments, a skin treatment system comprises a handpiece configured to contact a skin surface being treated comprises at least one fluid source configured to contain a treatment fluid; a handpiece, a fluid conduit placing the handpiece in fluid communication with the at least one fluid source, and a thermal conditioning assembly, the thermal conditioning assembly being configured to selectively heat and/or cool treatment fluid being transferred from the at least one fluid source to the handpiece, wherein the thermal conditioning assembly is located within the at least one fluid source.

According to some embodiments, a skin treatment system comprises a handpiece configured to contact a skin surface being treated comprises at least one fluid source configured to contain a treatment fluid; a handpiece, a fluid conduit placing the handpiece in fluid communication with the at least one fluid source, and a thermal conditioning assembly, the thermal conditioning assembly being configured to selectively heat and/or cool treatment fluid being transferred from the at least one fluid source to the handpiece, wherein the thermal conditioning assembly is located between an outlet of the at least one fluid source and the fluid conduit.

According to some embodiments, the thermal conditioning assembly is configured to heat and/or cool treatment fluid convectively. In some arrangements, the thermal conditioning assembly is configured to heat and/or cool treatment fluid conductively.

According to some embodiments, the skin treatment system further comprises at least one thermal conditioning module positioned within the handpiece, said at least one thermal conditioning module configured to heat and/or cool fluid entering the handpiece from the fluid conduit.

According to some embodiments, the thermal conditioning assembly comprises an ice bath. In some embodiments, the thermal conditioning assembly comprises a fluid bath to heat or cool the treatment fluid.

According to some embodiments, treatment fluid is heated or cooled conductively using the thermal conditioning module in the handpiece. In some embodiments, treatment fluid is heated or cooled convectively using the thermal conditioning module in the handpiece.

According to some embodiments, the thermal conditioning module comprises an electric heater. In some embodiments, the thermal conditioning module comprises a thermoelectric device or another type of heat pump.

According to some embodiments, a handpiece for use in a skin treatment procedure comprises a main body, a tip positioned along a distal end of the main body, and at least one fluid conduit configured to fluidly connect the tip to a fluid source. According to some embodiments, the handpiece further comprises a thermal conditioning assembly or module, the thermal conditioning assembly or module being configured to selectively heat and/or cool treatment fluid being transferred to the tip. In some embodiments, treatment fluid is heated or cooled conductively. In some embodiments, treatment fluid is heated or cooled convectively. In some embodiments, the thermal conditioning assembly or module comprises an electric heater. In some embodiments, the thermal conditioning assembly or module comprises a thermoelectric device or another type of heat pump.

According to some embodiments, a handpiece for use in skin treatment procedure comprises a main body, a tip positioned along a distal end of the main body, and a thermal conditioning assembly or module, the thermal conditioning assembly or module being configured to selectively heat and/or cool treatment fluid being transferred to the tip.

According to some embodiments, treatment fluid is heated or cooled conductively. In some embodiments, treatment fluid is heated or cooled convectively. In some embodiments, the thermal conditioning assembly or module comprises an electric heater. In some arrangements, the thermal conditioning assembly or module comprises a thermoelectric device or another type of heat pump.

According to some embodiments, a method for treating skin comprises performing a skin treatment procedure using a tip of a handpiece, the handpiece comprising a main body and the tip, wherein the tip is positioned at or near a distal end of the main body, and selectively heating and/or cooling a treating fluid being transferred to the tip using a thermal conditioning assembly or module of the handpiece.

According to some embodiments, treatment fluid is heated or cooled conductively. In some embodiments, treatment fluid is heated or cooled convectively. In some embodiments, the thermal conditioning assembly or module comprises an electric heater. In some arrangements, the thermal conditioning assembly or module comprises a thermoelectric device or another type of heat pump.

According to some embodiments, a handpiece for use in skin treatment procedure comprises a main body; a tip positioned along a distal end of the main body; and a thermal conditioning assembly or module, the thermal conditioning assembly or module being configured to selectively heat and/or cool treatment fluid being transferred to the tip; wherein the thermal conditioning assembly or module includes a fluid inlet and a fluid outlet, wherein a fluid pathway extending between the fluid inlet and the fluid outlet includes at least one turn; wherein the thermal conditioning assembly or module includes a heating or cooling component and at least one heat transfer component adjacent the heating or cooling component at or near the fluid pathway; and wherein the at least one heat transfer component is configured to selectively heat or cool treatment fluid passing through the fluid pathway when the heating or cooling component is electrically energized or activated.

According to some embodiments, treatment fluid is heated or cooled conductively; wherein the thermal conditioning assembly or module is configured to both heat and cool treatment fluid; and wherein the at least one heat transfer component at least partially extends into the fluid pathway.

According to some embodiments, treatment fluid is heated or cooled conductively.

According to some embodiments, treatment fluid is heated or cooled convectively.

According to some embodiments, a thermal conditioning assembly or module is configured to both heat and cool treatment fluid.

According to some embodiments, a thermal conditioning assembly or module includes an electric heater.

According to some embodiments, a thermal conditioning assembly or module includes a thermoelectric device or another type of heat pump.

According to some embodiments, a method for treating skin comprises performing a skin treatment procedure using a tip of a handpiece, the handpiece including a main body and the tip, wherein the tip is positioned at or near a distal end of the main body; and selectively heating and/or cooling a treatment fluid being transferred to the tip using a thermal conditioning assembly or module of the handpiece; wherein selectively heating and/or cooling a treatment fluid includes using a thermal conditioning assembly or module; wherein the thermal conditioning assembly or module includes a fluid inlet and a fluid outlet, wherein a fluid pathway extending between the fluid inlet and the fluid outlet includes at least one turn; wherein the thermal conditioning assembly or module includes a heating or cooling component and at least one heat transfer component extending between the heating or cooling component at the fluid pathway; and wherein the at least one heat transfer component is configured to selectively heat or cool treatment fluid passing through the fluid pathway when the heating or cooling component is electrically energized.

According to some embodiments, treatment fluid is heated or cooled conductively. According to some embodiments, a thermal conditioning assembly or module is configured to both heat and cool treatment fluid.

According to some embodiments, treatment fluid is heated or cooled conductively.

According to some embodiments, treatment fluid is heated or cooled convectively.

According to some embodiments, a thermal conditioning assembly or module is configured to both heat and cool treatment fluid.

According to some embodiments, a thermal conditioning assembly or module includes an electric heater.

According to some embodiments, a thermal conditioning assembly or module includes a thermoelectric device or another type of heat pump.

According to some embodiments, a skin treatment system comprises a handpiece configured to contact a skin surface being treated, the skin treatment system including: at least one fluid source configured to contain a treatment fluid; a handpiece; a fluid conduit placing the handpiece in fluid communication with the at least one fluid source; and a thermal conditioning assembly, the thermal conditioning assembly being configured to selectively heat and/or cool treatment fluid being transferred from the at least one fluid source to the handpiece; wherein the thermal conditioning assembly is located within the at least one fluid source.

According to some embodiments, a thermal conditioning assembly is configured to heat and/or cool treatment fluid convectively.

According to some embodiments, a thermal conditioning assembly is configured to heat and/or cool treatment fluid conductively.

According to some embodiments, a skin treatment system further includes at least one thermal conditioning module positioned within the handpiece, said at least one thermal conditioning module configured to heat and/or cool fluid entering the handpiece from the fluid conduit.

According to some embodiments, a thermal conditioning assembly includes an ice bath.

According to some embodiments, a thermal conditioning assembly includes a fluid bath to heat or cool the treatment fluid.

According to some embodiments, a treatment fluid is heated or cooled conductively using the at least one thermal conditioning module in the handpiece.

According to some embodiments, a treatment fluid is heated or cooled convectively using the at least one thermal conditioning module in the handpiece.

According to some embodiments, at least one thermal conditioning module includes an electric heater.

According to some embodiments, at least one thermal conditioning module includes a thermoelectric device or another type of heat pump.

According to some embodiments, a thermal conditioning assembly is configured to both heat and cool treatment fluid being transferred from the at least one fluid source to the handpiece.

According to some embodiments, a skin treatment system includes a handpiece configured to contact a skin surface being treated, the skin treatment system including: at least one fluid source configured to contain a treatment fluid; a handpiece; a fluid conduit placing the handpiece in fluid communication with the at least one fluid source; and a thermal conditioning assembly, the thermal conditioning assembly being configured to selectively heat and/or cool treatment fluid being transferred from the at least one fluid source to the handpiece; wherein the thermal conditioning assembly is located between an outlet of the at least one fluid source and the fluid conduit.

According to some embodiments, a thermal conditioning assembly is configured to heat and/or cool treatment fluid convectively.

According to some embodiments, a thermal conditioning assembly is configured to heat and/or cool treatment fluid conductively.

According to some embodiments, a system further includes at least one thermal conditioning module positioned within the handpiece, said at least one thermal conditioning module configured to heat and/or cool fluid entering the handpiece from the fluid conduit.

According to some embodiments, a thermal conditioning assembly includes an ice bath.

According to some embodiments, a thermal conditioning assembly includes a fluid bath to heat or cool the treatment fluid.

According to some embodiments, treatment fluid is heated or cooled conductively using the at least one thermal conditioning module in the handpiece.

According to some embodiments, treatment fluid is heated or cooled convectively using the at least one thermal conditioning module in the handpiece.

According to some embodiments, at least one thermal conditioning module includes an electric heater.

According to some embodiments, at least one thermal conditioning module includes a thermoelectric device or another type of heat pump.

According to some embodiments, a thermal conditioning assembly is configured to both heat and cool treatment fluid being transferred from the at least one fluid source to the handpiece.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 schematically illustrates a handpiece or handheld device configured to be used for skin treatment;

FIG. 2A schematically illustrates a flow diagram for a skin treatment system according to one embodiment;

FIG. 2B schematically illustrates a flow diagram for a skin treatment system according to another embodiment;

FIG. 3 illustrates one embodiment of a tower or console of a skin treatment system comprising a manifold assembly;

FIG. 4 schematically illustrates a cross-sectional schematical view of a handpiece comprising a thermal conditioning module or assembly according to one embodiment;

FIG. 5 schematically illustrates a partially transparent perspective view of a thermal conditioning module or assembly for a handpiece or other portion of a skin treatment device according to one embodiment;

FIGS. 6A and 6B illustrate different views of a thermal conditioning module or assembly for a handpiece or other skin treatment device according to one embodiment;

FIGS. 7A to 7C illustrate different views of a thermal conditioning module or assembly for a handpiece or other skin treatment device according to one embodiment;

FIG. 8 illustrates a plate or other component of a thermal conditioning module or assembly that comprises one or more fluid channels or passages according to one embodiment;

FIGS. 9A and 9B schematically illustrate embodiments of example fluid flow patterns created using channels or other passages in a thermal conditioning module or assembly for a handpiece or other skin treatment device according;

FIG. 10 schematically illustrates one embodiment of a thermal conditioning system or module for a skin treatment system;

FIG. 11 schematically illustrates one embodiment of a thermal conditioning system or module for a skin treatment system; and

FIG. 12 schematically illustrates one embodiment of a thermal conditioning system or module for a skin treatment system.

### DETAILED DESCRIPTION

Although the various embodiments of a handpiece assembly and/or other components or portions of a skin treatment system have specific relevance to a skin treatment system, the features, advantages and other characteristics disclosed herein may have direct or indirect applicability in other applications, such as, for example, medical devices, mechanical devices and/or the like.

Several embodiments of the inventions disclosed herein are particularly advantageous because they include one, several or all of the following benefits: provide a handpiece for use in a skin treatment system that provides one or more features that enhance the execution of a corresponding treatment procedure, facilitate a treatment procedure for the aesthetician or other user, provide a more comfortable and pleasant experience for the subject that is undergoing the skin treatment procedure and/or the like; improve the efficacy, safety and/or other aspects of a skin treatment procedure; and selectively heat and/or cool treatment materials and/or other fluids being delivered to the tip and the subject's skin surface.

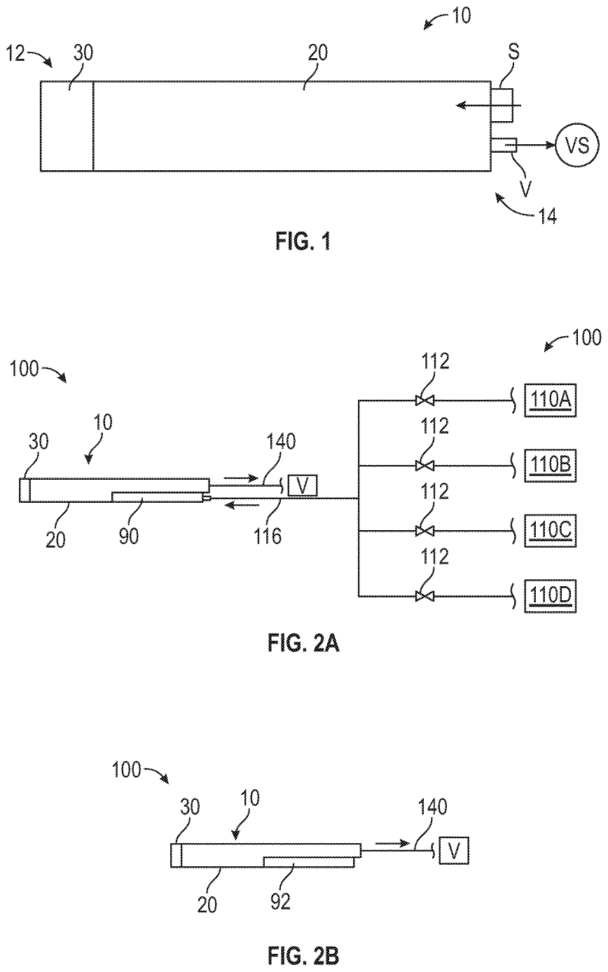

FIG. 1 illustrates one embodiment of a handpiece or other device 10 configured to be used for skin treatment. As shown, the handpiece or device 10 can include a main body or main body portion 20 and a tip 30 secured along the distal end 12 of the main body 20. In some embodiments, the tip 30 comprises a separate component or device that is configured to removably secure to the main body 20. However, in other arrangements, the tip 30 and the main body 20 form a unitary, monolithic and/or non-separable configuration, as desired or required.

The handpiece can be adapted to selectively, at least partially, heat and/or cool treatment fluids at least as they are being transferred to the distal end 12 of the device 10 (e.g., to or near the tip 30). In some embodiments, such thermal conditioning (e.g., heating and/or cooling) of fluids and/or other treatment materials being delivered to a subject's skin via a handpiece is performed solely in and/or by the handpiece. However, in other arrangements, as discussed herein, thermal conditioning of treatment materials can be performed at and/or by other components of a skin treatment system (e.g., a console or tower, a separate device or component, etc.), either in lieu of or in addition to at/by the handpiece. The ability to perform such heating and/or cooling of treatment materials within the handpiece or device can enhance the efficacy of a particular skin treatment procedure, can improve the comfort level and/or overall feel to the subject who is undergoing a skin treatment procedure and/or provide one or more additional benefits or advantages. In addition, the device 10 can include one or more electric/electrical, electronic, mechanical, electromechanical and/or other components or features to assist with the implementation of one or more of the features described herein into its design. Such components or features can include, without limitation, wiring, batteries or other power-related components, chips, boards, buttons, switches or other controllers, control units or processors, memory components, valves, sensors, housings, other enclosures and/or the like, as desired or required.

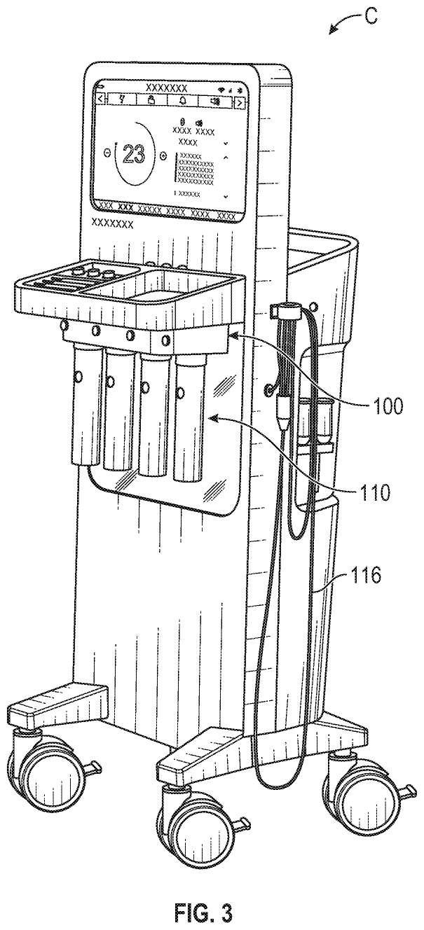

With continued reference to FIG. 1, for any of the embodiments disclosed herein or equivalents thereof, a handpiece or device 10 can be configured to transfer treatment materials and other fluids from one or more containers or other fluid sources S (e.g., a vial (not shown), a cartridge that is in fluid communication with a larger manifold or fluid distribution system (e.g., such as the system depicted in FIG. 3), etc.) through one or more conduits (also not shown) passing, at least partially, through an interior of the main body 20 of the device 10. The device 10 can be configured to be placed in fluid communication with a vacuum or suction source VS (e.g., via one or more vacuum conduits V) and a waste container. The handpiece 10 can be configured such that the creation or application of vacuum or suction creates, at least in part, a vacuum or suction force along the tip 30.

The application of suction or vacuum at, along and/or near the tip 30 or other portion of or near the distal end 12 of the device 10 can assist with removing spent fluid, debris and/or other waste (e.g., sebum, dirt, oils, exfoliated skin or other tissue of the subject being treated, etc.) away from the working end of the device 10 during use. Further, in some arrangements, the application of vacuum or suction along the working end (e.g., via activation of a vacuum or suction source VS) can help draw treatment materials and/or other fluids (e.g., acidic solutions, serums, etc.) to the working end. For instance, treatment fluids can be transferred from a fluid source S (e.g., a vial, a manifold or fluid distribution system, etc.) to the tip 30 when at least a partial seal is formed (or substantially formed) between the tip 30 and a portion of the skin surface being treated. Under such circumstances, the application of vacuum or suction can help draw treatment fluids through one or more ports of the tip and via one or more fluid conduits placing the fluid source S in fluid communication with the tip 30 (e.g., while simultaneously removing waste from the tip to a waste container or area).

For any of the embodiments disclosed herein, the tip 30 or other portion of the distal end 12 of the device 10 adapted to contact skin tissue can be configured to at least partially abrade, exfoliate and/or otherwise remove skin tissue (e.g., when the device is in use). In some embodiments, the tip 30 and/or other portion of or near the distal end 12 of the device 10 comprises one or more features (e.g., edges, protruding components, ridges, etc.) that assist with exfoliation or other skin removal. For example, such features can help remove skin when the tip 30 or other portion of the distal end 12 of the device 10 is moved (e.g., translated) relative to the targeted skin surface (e.g., in a direction parallel or substantially parallel to the adjacent skin surface being treated). In some arrangements, the efficacy and/or extent of exfoliation or other skin removal is enhanced by the application of suction or vacuum along the working end (e.g., tip, distal end, etc.) of the device.

According to certain embodiments, as schematically illustrated in FIG. 2A, one or more bottles or other containers 110A, 110B, 110C, 110D can be placed in fluid communication with a manifold assembly or other fluid distribution assembly or system 100. In some arrangements, as illustrated in FIG. 2A, one or more valves or other flow control devices or components 112 can be positioned between each fluid source (e.g., a bottle) and a main delivery conduit or other fluid line 116 that places the manifold assembly 100 in fluid communication with a handpiece 10. The valves or other flow control devices 112, which may include 2-way valves, can be configured to regulate the flow of treatment materials from one or more of the bottles or other containers 110A, 110B, 110C, 110D to the main delivery conduit 116, and thus, to the handpiece or other skin-interface device 10.

With continued reference to FIG. 2A, the handpiece 10 can be placed in fluid communication with a suction source (e.g., vacuum pump, not shown) using one or more vacuum or suction conduits or other fluid lines 140. In the arrangements disclosed herein, by way of example, the manifold assembly or fluid distribution system or component 100 comprises a total of four fluid branches. However, a system can comprise more or fewer fluid branches (e.g., 1, 2, 3, 5, 6, 7, 8, more than 8, etc.), as desired or required by a particular application or use. Further, one or more of the bottles or other containers 110A, 110B, 110C, 110D that are positioned within the manifold assembly 100 can include a cleaning, flushing and/or rinsing solution (e.g., for the periodic flushing and cleaning of the manifold assembly).

According to certain embodiments, one or more of the fluid conduits of the manifold system illustrated in FIG. 2A are configured to transfer a serum, other treatment fluids and/or treatment materials and/or the like to a handpiece or other downstream location. Alternatively, however, one or more of the conduits can be configured to receive water (e.g., distilled, tap water, filtered, etc.), saline, other dilutants or dissolvents, other fluids and/or the like to the handpiece assembly 10. Such fluids can be adapted to at least partially contact and dissolve, dilute, liquefy, soften and/or otherwise mix with one or more solids, gels and/or other materials positioned within or on various surfaces or portions of the handpiece 10 (e.g., tip 30 or distal end 12). This can provide a convenient method of providing one or more materials at the skin-tip interface and/or any other location where such materials are desired or required. Further, such embodiments can provide certain benefits to the environment, as the carbon footprint of producing, transporting, using, disposing, etc. related to such consumable products is advantageously reduced. For instance, fewer disposable treatment material containers are needed.

Further, as illustrated schematically in FIG. 2B and discussed in greater detail herein, for any device or system disclosed herein or equivalent thereof, the device or system can be configured to permit a user (e.g., an aesthetician, a cosmetician, a dermatologist, etc.) to (1) connect a handpiece 10 to a manifold assembly or fluid distribution system 100 (e.g., using a “dummy” cartridge 90, as shown in FIG. 2A), and/or (2) insert an actual fluid or treatment material-containing cartridge 92 directly into the handpiece assembly (e.g., a recess of the handpiece assembly (as shown in FIG. 2B). These two options can be used interchangeably to perform a particular treatment procedure or protocol or series of procedures or protocols, as desired or required.

One embodiment of a tower or console C that includes a manifold assembly 100 is illustrated in FIG. 3. As shown, the manifold assembly 100 can be configured to receive one or more bottles or other containers 110. In the illustrated arrangement, the manifold assembly 100 is adapted to receive up to four bottles or containers 110. However, as noted herein, in other arrangements, a manifold assembly 100 can receive less than four (e.g., 1, 2 or 3) or greater than four (e.g., 5, 6, 7, 8, more than 8) bottles or containers 110, as desired or required. A fluid conduit or other passage 116 can fluidly couple or connect the manifold assembly 100 to a handpiece or other handheld device 10. In some embodiments, a tower or console C can also include one or more waste containers or receptacles for collecting spent fluids, exfoliated skin, materials removed from a subject's skin during a procedure and other waste materials.

According to some embodiments, a handpiece or other device for skin treatment 10 is configured to selectively heat and/or cool treatment materials and/or other fluids being delivered to a skin surface using such a handpiece or other device. Thermal conditioning or manipulation (e.g., heating and/or cooling) of treatment materials and other fluids being delivered to the skin surface (e.g., water) can provide one or more benefits or advantages. For instance, providing heated fluid (e.g., fluid having a temperature above ambient temperature, fluid heating to a particular threshold or range, etc.) can enhance the efficacy of a treatment procedure. In some embodiments, heat can help open the pores of a subject's skin, thereby allowing any treatment materials or fluids delivered to the skin surface to penetrate deeper into the skin surface. Such deeper penetration into skin by treatment materials can provide a better result for the skin treatment procedure or protocol being performed. In some embodiments, the application or removal of heat (e.g., heating, cooling) of skin tissue (e.g., by directly heating or cooling skin tissue, by heating or cooling a treatment fluid or other materials being delivered to the skin, etc.) can help activate certain compounds that have been delivered to the subject (e.g., topically, via an ingestible product, etc.).

In some embodiments, thermal conditioning can also enhance the comfort and feel of the subject that is undergoing a treatment procedure. For example, heating and/or cooling effects via heated and/or cooled treatment fluids delivered to the skin can stimulate a pleasing effect to the subject undergoing treatment. Further, the heating and/or cooling of treatment fluids can extend the residence time of such fluids within the skin. One or more additional benefits and/or advantages relating to thermal conditioning of skin tissue (e.g., directly, indirectly, etc.) may exist and/or may be appreciated at a later time, and the various embodiments disclosed herein can assist with advancing such benefits and/or advantages.

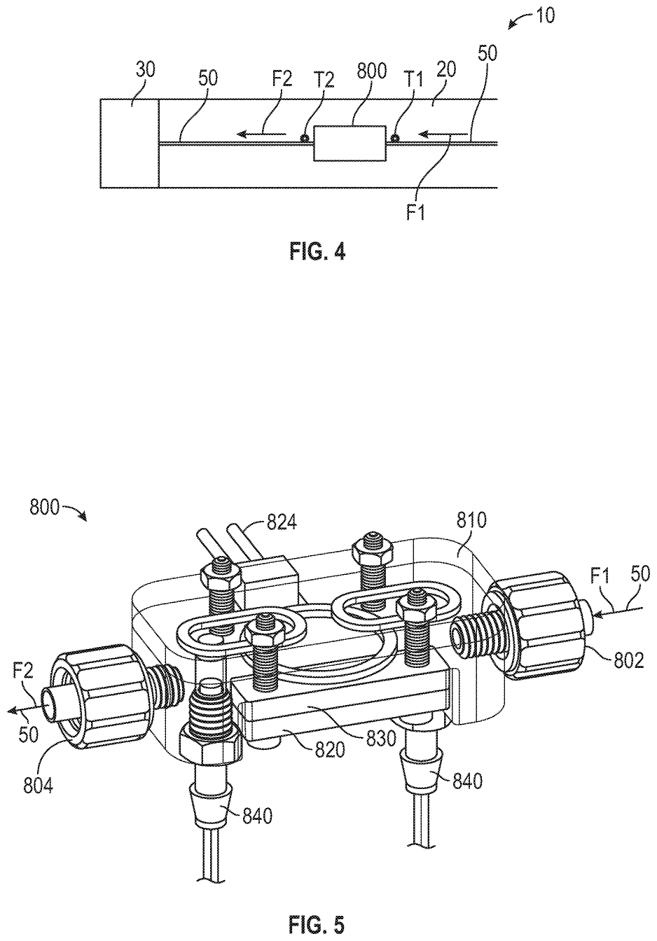

FIG. 4 illustrates a schematic of one embodiment of a handpiece 10 comprising a thermal conditioning module 800 configured to selectively heat and/or cool fluid being delivered to the tip or distal end 30 of a handpiece or other skin treatment device 10. As shown, the thermal conditioning module 800 can be positioned entirely within an interior portion of the handpiece or other device 10 (e.g., within an interior of the main portion 20 of the handpiece 10). However, in other arrangements, one or more portions of the thermal conditioning module can be positioned at least partially (e.g., partially, completely, etc.) on the outside of the main portion 20 (e.g., along an exterior) and/or other non-interior portion of the device 10. In some embodiments, for example, the thermal conditioning module 800 can be positioned completely or partially within and/or on a tip 30 or other component. In some arrangements, at least one thermal conditioning module is positioned both within a handpiece and along the exterior or outside of the handpiece.

As noted above, a thermal conditioning module 800 can be configured to selectively only heat treatment fluids and/or other materials, to selectively only cool them, or to selectively heat and cool them, as desired or required. In some embodiments, heating or cooling entails or includes (and may mean) performing some level of heat transfer to raise or lower the temperature of a treatment material or other fluid (e.g., relative to its stored temperature, relative to ambient temperature, relative to some baseline temperature (e.g., 20 degrees C., 70 degrees Fahrenheit, etc.), etc.).

In some embodiments, the thermal conditioning module 800 is configured to heat and/or cool a fluid using conductive heat transfer. However, in other arrangements, convective heat transfer can be used, either in lieu of or in addition to conductive heat transfer. Accordingly, the thermal conditioning module 800 can include one or more components to accomplish the necessary heating, cooling, and associated heat transfer with fluids being delivered to a subject's skin. Such components can include, without limitation, a positive temperature coefficient (PTC) heater, other types of electric heaters, other heaters, Peltier or thermoelectric devices, other heat pump devices and/or the like.

With continued reference to FIG. 4, the thermal conditioning module 800 can be positioned at least partially within or near the flow of the treatment material or fluid being transferred through one or more fluid conduits 50 of the handpiece 10. Therefore, at least a portion of the fluid being transferred toward the tip 30 can be passed through, along or near at least a portion of the thermal conditioning module 800 (e.g., to accomplish a necessary or desired level of heat transfer to or from the fluid). In some embodiments, treatment material or fluid flowing toward or into the module 800 (generally represented by arrow F1 in FIG. 4) can include a first temperature T1 (e.g., an inlet temperature), whereas treatment material or fluid flowing away from or out of the module 800 (generally represented by arrow F2) can include a second temperature T2 (e.g., an outlet temperature).

In embodiments where the thermal conditioning module 800 is activated to heat fluid passing through, along or near it, the outlet temperature, T2, is higher than the inlet temperature, T1. In contrast, if the thermal conditioning module 800 is configured to cool fluid, T2 will be lower than T1. The level of heating and/or cooling can be controlled automatically (e.g., by the handpiece, another system to which the handpiece is operatively coupled, etc.) and/or manually (e.g., by the aesthetician, other professional or user, etc.). For example, in a hybrid system, an aesthetician can alter the level of thermal conditioning (e.g., heating, cooling, etc.) within a specific temperature range. Thus, the system can automatically ensure that any attempted operational changes attempted by a user remain within certain upper and/or lower thresholds or limits.

In some arrangements, the module 800 and/or other portions or components of the handpiece 10 comprise one or more temperature sensors 840 to provide the necessary feedback to a control system. This can help ensure that the level of heating and/or cooling can be adjusted (e.g., in real time) to ensure that the desired or required level of thermal conditioning is achieved on the treatment material or fluid being transferred by the handpiece 10. A control system can include, for example and without limitation, the following: one or more processors, one or more memories, one or more sensors, one or more wired or wireless connections, devices, components and/or protocols, one or more controllers, one or more inputs, one or more outputs and/or the like.

According to some embodiments, the thermal conditioning module can be sized, designed and/or otherwise configured to perform a desired level of heat transfer to a fluid stream so that fluid and/or other treatment material exiting the module (and thus, passing to the tip and skin surface) can be modified within a particular temperature range and/or above or below particular thresholds. For example, the module 800 can be configured to be able to provide the fluid to the skin surface at a temperature range of 5 to 95 degrees Celsius (e.g., 5 to 60, 5 to 10, 10 to 20, 20 to 30, 30 to 40, 40 to 50, 50 to 60, 30 to 60, 45 to 55, 55 to 60, 60 to 70, 70 to 80, 80 to 90 degrees, temperature values between the foreign values or ranges, etc.). In other embodiments, the module 800 can be configured to be able to provide the fluid to the skin surface at a temperature above 95 degrees Celsius (e.g., 95 to 100, 100 to 105, above 105 degrees Celsius), as desired or required. As mentioned above, a desired level or range for fluid temperature being delivered to a subject's skin can be adjusted by heating and/or cooling the fluid (e.g., using one or more components of a handpiece and/or other components or portions of a skin treatment system).

As noted herein, the thermal conditioning module 800 can include one or more Peltier or thermoelectric devices, other heat pumps and/or any other heating or cooling device. Depending on the type of thermal conditioning module(s) 800 that is/are incorporated into a handpiece 10, one or more heat exchange members (e.g., pins, fins, etc.) may need to be incorporated into the thermal conditioning module(s) and/or used with the thermal conditioning module(s). Such heat exchange members can be configured to work with or otherwise accommodate (e.g., to move heat to or away from one or more portions of the thermal conditioning module where the heating and/or cooling of treatment fluid occurs) any type of fluid (e.g., fluids that are likely to contact the heat exchange members), including, for example, air (e.g., ambient air), other gases, water, other fluid (e.g., any waste fluid being removed from the skin surface being treated), etc. to dissipate or otherwise move heat from the heat exchange members. Therefore, in some embodiments, the heat exchange members (and the thermal conditioning modules for which the heat exchange members are being used) can be air-cooled, water-cooled, liquid-cooled, fluid-cooled and/or the like.

One embodiment of a thermal module 800 is illustrated in FIG. 5. As shown, fluid and/or other treatment material F1 can enter the module 800 from a first end. Such fluid F2 can exit the module from a second, opposite end after it has passed through, along or near the module and has undergone the desired or required amount of heating and/or cooling. In some embodiments, the module 800 can include inlet and/or outlet couplings and/or other features or connections 802, 804 to facilitate connection of the module 800 to one or more fluid conduits 50 and/or other fluid components of the handpiece or device 10.

With continued reference to FIG. 5, the thermal conditioning module 800 can include a housing 810 that at least partially encloses one or more internal spaces or portions. In some embodiments, treatment materials and/or other fluids can be configured to enter into, at least partially, an area enclosed by the housing 810. Heating and/or cooling of fluids and/or other treatment materials can occur, at least partially, within the housing 810 of the module 800. The thermal conditioning module 800 can include one or more heating and/or cooling elements 820 that are configured to selectively transfer heat to and/or from the fluid passing through, along or near (e.g., within the housing 810 of) the module 800. As noted above, such heating and/or cooling elements 820 can include, for example and without limitation, electric heaters (e.g., resistive heating member), Peltier or thermoelectric devices, other heat pumps and/or any other heating or cooling device.

As illustrated in FIG. 5, the thermal conditioning module 800 can include one or more other components, such as, for example, a heatsink or other heat transfer component 830 (e.g., a conductive block for conductive heating or cooling, fins or other heat transfer members for convective heating or cooling, etc.), thermistors or other temperature sensors 840, PCBs, chips, wiring, cables and/or other electrical conductors 824 (e.g., for supplying electrical current to the heating and/or cooling element 820), processors, other electrical or electronic members or components and/or the like.

As noted herein, having the ability to selectively heat and/or cool a fluid being transferred by a handpiece or other device 10 can provide certain benefits and advantages to a treatment procedure. For example, the efficacy and ultimate result of a procedure can be enhanced (e.g., when or by using fluid delivery, microdermabrasion and/or the like) if the fluid is maintained at a particular temperature or within a particular temperature range. Such benefits can be related to the creation of a desired thermal conditioning (e.g., heating, cooling, etc.) of the subject's skin surface (e.g., opening or closing of pores, creating a numbing effect, creating another anatomical effect, etc.), the impact of thermal conditioning on the treatment materials themselves (e.g., activating or deactivating certain ingredients of a treatment fluid, extending a life or effect of one or more ingredients, etc.) and/or the like. Further, such thermal conditioning of fluids can improve the feel, comfort and enjoyment level of the subject undergoing the treatment procedure and/or provide one or more additional advantages or benefits.

In some embodiments, a temperature on, along or near the skin surface of a subject can be measured before, during and/or after a treatment procedure. In some arrangements, the handpiece or other handheld device 10 is configured to measure such a temperature on, along or near the skin surface of a subject. Accordingly, one or more sensors (e.g., temperature sensors, contact sensors, etc.) can be included along the tip and/or other portion of the handpiece. In other embodiments, such a temperature can be obtained using any other device, system and/or technology. Regardless of how, where and/or when temperature is measured, temperature detection can include detecting the temperature of a skin surface, the temperature of fluid passing by or near the module and/or any other temperature measurements obtained.

As discussed in greater detail herein (e.g., in connection with illustrated embodiments or variations thereof), heating and/or cooling of a treatment fluid and/or other treatment material can be accomplished, at least in part, by transferring heat to and/or from said fluid/material. Such transfer can occur at least partially within a thermal conditioning module, for example, by using conductive and/or convective heat exchange components and methods. In some embodiments, as noted herein, heat transfer to and/or from treatment fluid and/or other treatment material can include the use of one or more heat transfer members (e.g., pins, fins, etc.). However, in alternative embodiments, heat transfer to and/or from a treatment fluid can occur without the use of heat transfer members. For instance, heat transfer can occur via direct thermal contact between a heating or cooling element and fluid being transferred at least partially through a thermal conditioning module.

For any of the embodiments disclosed herein, one or more temperature sensors (e.g., thermistors) can be included in a thermal conditioning module 800. The use of such sensors 840 can help determine the amount of heating and/or cooling that is being provided to a treatment fluid using the thermal conditioning module. For example, in the embodiment depicted in FIG. 5, as well as other embodiments illustrated herein, the module 800 can include inlet and outlet temperature sensors 840. A handpiece can include one or more additional sensors or sensor modules, either in addition to or in lieu of a thermal conditioning module 800.

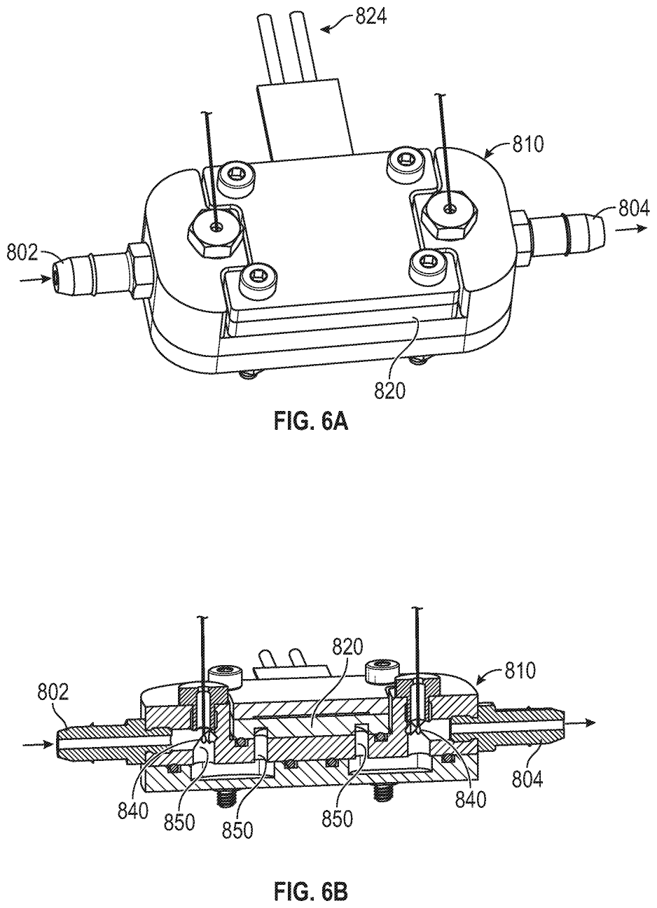

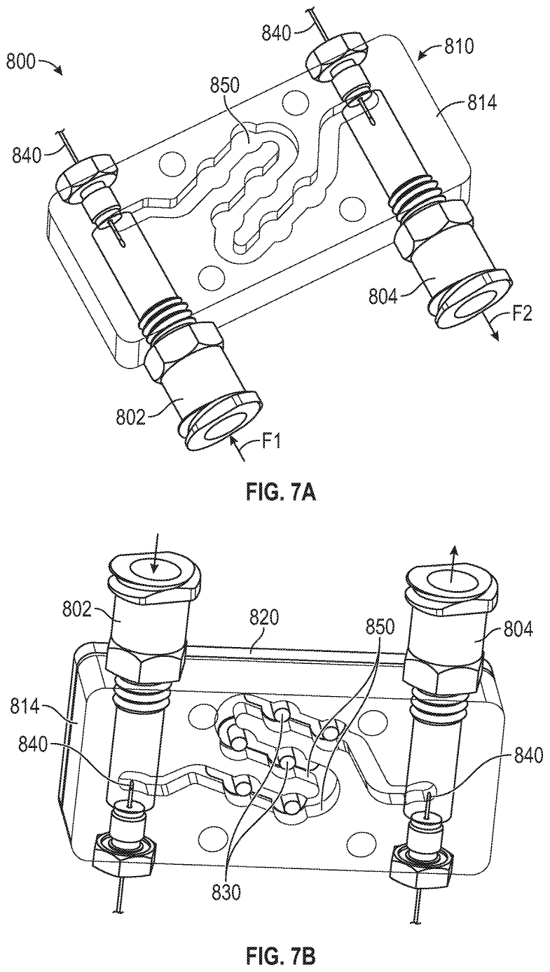

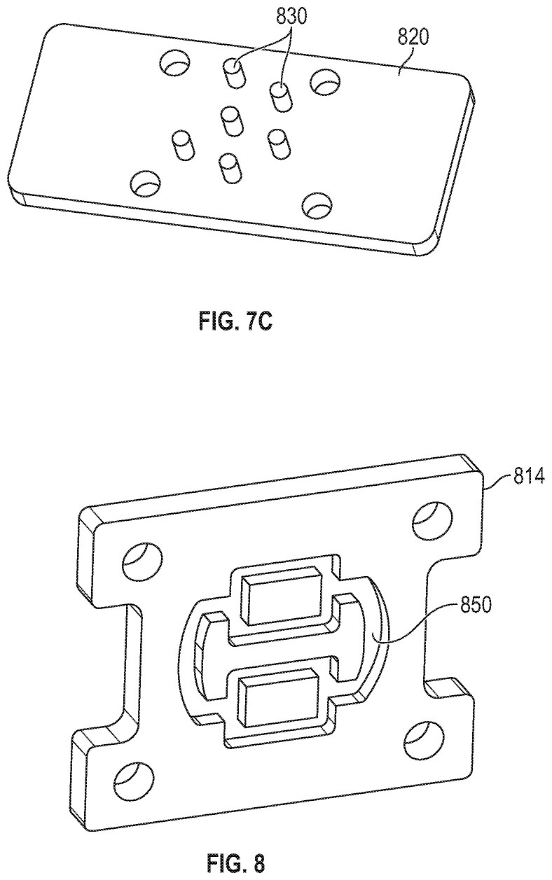

For any of the embodiments disclosed herein, one or more fluid flow channels or passages 850 are created or included within the thermal conditioning module 800. Such channels or other passages 850 can include recesses or other channels formed within a plate and/or other component of the module. Such recesses or channels can be enclosed, in some embodiments, by securing another plate or member adjacent the recesses or channels to create the passages or channels 850. However, fluid channels or passages can be created using any other technique or method, as desired or required. FIGS. 6A through 7B depict example embodiments of a fluid flow channel or passage 850 within a thermal conditioning module 800.

Further, for any of the thermal conditioning module configurations disclosed herein or equivalents thereof, the module 800 can include inlet and outlet couplings 802, 804. Such couplings 802, 804 can include standard or non-standard couplings, as desired or required by a particular application or use. In some embodiments, such couplings 802, 804 comprise luer or other threaded couplings. Such couplings, regardless of their exact design or configuration, can be sized, shaped and otherwise adapted to connect to the fluid delivery network of the handpiece or other handheld device.

FIGS. 6A and 6B illustrate different views of another embodiment of a thermal conditioning module 800 that can be used to selectively heat and/or cool fluid being delivered to a skin surface using a handpiece or other handheld device. As shown, the module 800 can include one or more thermal conditioning (e.g., heating and/or cooling) elements, components, devices and/or the like 820. Such thermal conditioning elements 820 can be electrically energized using one or more wires or other electrical conductors 824 that couple the module 800 with an electrical energy source (e.g., battery, other power source, etc., not shown).

With continued reference to FIGS. 6A and 6B, the module 800 can comprise one or more thermal conditioning elements or components 820 that are directly or indirectly adjacent to the channels 850 through which treatment fluid passes. Thus, heat transfer to and/or from the treatment fluid passing through the fluid channels or passages 850 can be accomplished, at least in part or entirely, via conduction and/or convection. As illustrated, the thermal conditioning elements or components 820 can be in direct contact with the fluid being transferred. However, in other embodiments, one or more intermediate members, elements or components can be included. In such arrangements, direct contact does not exist between the thermal conditioning element (e.g., heating and/or cooling device, component, member, etc.) and the fluid and/or material being heated and/or cooled.

Another embodiment of a thermal conditioning module 800 that can be incorporated into a handpiece and/or other portion of a skin treatment system is illustrated in FIGS. 7A to 7C. As with other embodiments disclosed herein, the depicted module 800 can be used to selectively heat and/or cool fluid and/or other treatment materials being delivered to a skin surface using a handpiece or other handheld device. As shown, the module 800 can include one or more thermal conditioning (e.g., heating and/or cooling) elements, components, devices and/or the like 820. Such thermal conditioning elements 820 can be electrically energized or activated using one or more wires or other electrical conductors that couple the module 800 with an electrical energy source (e.g., battery, other power source, not shown).

With continued reference to FIGS. 7A to 7C, the module 800 can comprise one or more thermal conditioning elements or components 820 that are directly or indirectly adjacent to the channels 850 through which treatment fluid passes. Thus, heat transfer to and/or from the treatment fluid passing through the fluid channels or passages 850 occurs via conduction and/or convection. As illustrated, the thermal conditioning elements or components 820 can be in direct contact with the fluid being transferred. However, in other embodiments, one or more intermediate members, elements or components can be included. In such arrangements, direct contact does not exist or occur between the thermal conditioning element (e.g., heating and/or cooling device, component, member, etc.) and the fluid being heated and/or cooled.

As illustrated in FIGS. 7A and 7B, the fluid channels or passages 850 can include a non-linear shape or design. For example, in the depicted embodiment, the fluid channels or passages 850 for a serpentine shape or a S-shape (or other curved or non-linear shape). In some embodiments, the fluid channels or passages 850 are configured to make at least one gradual and/or abrupt turn. In the arrangement illustrated in FIGS. 7A and 7B, the fluid channels or passages 850 include three or at least three turns (e.g., 180 degrees or substantially 180 degrees) between the inlet and outlet couplings 802, 804, However, in other arrangements, the fluid channels or passages 850 can include more or fewer than three turns or other changes in direction (e.g., 1, 2, 4, 5, more than 5) with more or less severe (e.g., more or less acute) angles of turn, as desired or required. Further, such turns or other changes in direction can have a different angle, radius of curvature and/or one or more other characteristics or properties than shown in the embodiment of FIGS. 7A to 7C.

In some embodiments, incorporating one or more turns or other changes in direction in the fluid channels or passages in a thermal conditioning module 800 can provide one or more advantages or benefits to the corresponding handpiece and/or a treatment procedure that is performed using the handpiece. For example, a longer fluid flow path created within the thermal conditioning module 800 can help with accommodating a desired level of heat transfer to the treatment fluid (e.g., heating and/or cooling of the treatment fluid passing through and/or along the module 800) without increasing (e.g., unnecessarily increasing) the footprint or size of the module 800 (e.g., vis-à-vis a linear or substantially linear flow path). This can be helpful considering the limited space available within a handpiece or other skin treatment device into which the module will be incorporated.

Relatedly, the inclusion of a serpentine, tortuous and/or other non-linear flow path(s) for the treatment fluid and/or other treatment material passing through or near a thermal conditioning module 800 can also achieve higher levels of heating or cooling to the treatment fluid. The ability to achieve higher and/or lower temperatures (e.g., relative to ambient temperature, the storage temperature of the treatment fluid in a bottle, vial or other container that is in fluid communication with the handpiece, etc.) of the fluid delivered to the skin surface can allow for improved treatment results. This may be attributed to, for example and without limitation, deeper penetration and/or retention within a subject's skin, improved results from performing a procedure at a higher or lower temperature and/or the like.

With continued reference to FIG. 7B, a thermal module 800 can include a heating or cooling element or component 820 that, when electrically energized, is configured to become hot or cold (e.g., relative to its non-thermally conditioned temperature, the ambient temperature, etc.). As illustrated in FIG. 7C, the heating and/or cooling element or component 820 can include one or more pins or other heat transfer components 830. In some arrangements, such pins or other heat transfer features, members or components 830 are incorporated into the heating and/or cooling element or component 820. For example, the pins or other heat transfer features, members or components 830 and the heating and/or cooling element or component 820 can be manufactured as a single, unitary or monolithic member. In other arrangements, the pins or other heat transfer features, members or components 830 and the heating and/or cooling element or component 820 can be manufactured as separate members or components that are subsequently secured or otherwise operatively (e.g., directly or indirectly connected) to one another, as desired or required.

As illustrated in FIG. 7B, in some embodiments, when the heating and/or cooling element or component 820 is secured to other components and/or portions of the module 800 (e.g., the baseplate or other portion of the housing 810 or module), the pins or other heat transfer features, members or components 830 can extend, at least partially, into the fluid channels or pathways 850 of the module. For any of the embodiments disclosed herein or equivalents thereof, the fluid channels or pathways 850 are created, at least partially, within or along a plate 814 of the module. For example, such fluid channels or pathways 850 can include grooves or recesses that are formed or created with (or shortly after the formation of) the plate 814 of the module. In other embodiments, the fluid channels or pathways 850 are formed or created by removing material (e.g., cutting, etching, etc.) from a plate 814 or other component of the module 800. In some arrangements, the plate 814 that includes one or more fluid channels or pathways 850 also helps form at least a portion of the housing 810 of the module 800. However, the plate 814 can be separate and distinct from one or more components and/or portions of the housing 810 of the module 800, as desired or required by a particular application or use of a skin treatment system and/or any of its portions or components.

In some embodiments, when energized, the thermal module 800 can create a localized heat transfer effect or impact on any fluid and/or other material that is transferred through the channels or pathways 850, thereby selectively heating and/or cooling said fluid and/or material.

FIG. 8 illustrates another embodiment of a plate 814 or component of a thermal module 800 that includes one or more fluid channels or pathways 850 formed within its structure. Any variety of fluid pathway schemes or networks can be used to create a desired or required fluid flow pattern through or near a thermal conditioning module 800.

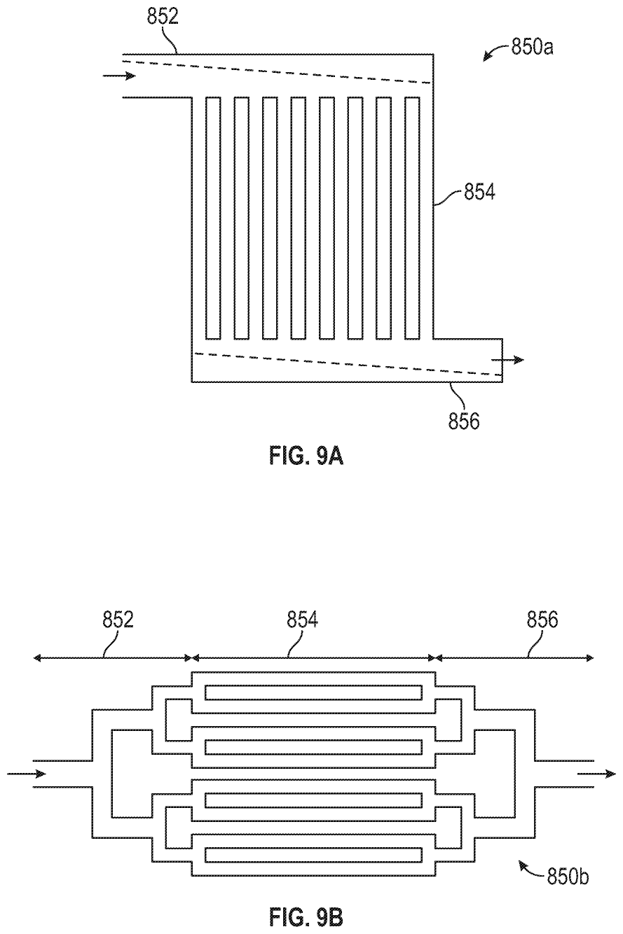

For example, as depicted schematically in FIG. 9A, a thermal conditioning module can include a fluid channel or pathway network 850A with a main inlet zone 852 and a main outlet zone 856 that is located generally opposite the main inlet zone 852 (e.g., along an opposite end or portion of the main inlet zone 852). As shown, a plurality of intermediate flow channels or pathways can be included in a transitional or intermediate zone 854. In the illustrated arrangement, the fluid channel or pathway network 850A can include a parallel configuration in which the various intermediate flow channels or pathways originate and/or terminate in a single main inlet zone 852 and/or main outlet zone 856.

In other embodiments, however, as shown in the embodiment of FIG. 9B, a thermal conditioning module can include a “series” or “manifold”/“header” configuration 850B where fluid passes from an inlet zone 852 to an outlet zone 856. For instance, in the illustrated arrangement, fluid entering into the inlet zone 852 is configured to be divided into smaller branches. In some embodiments, subsequent to dividing the flow into smaller branches (e.g., along a proximal portion of the intermediate zone 854), as depicted in FIG. 9B, the flow is combined into larger branches until it eventually exits through a main outlet zone 856.

The inclusion of smaller branches (e.g., along one or more fluid flow channels or pathways of an intermediate zone 854) can help with the heat transfer efficiency (e.g., to improve efficiency relative to embodiments without branches or similar features) between the pins or other heat transfer members or components 830 (see, e.g., FIG. 7C) and the fluid being transferred at least partially through or along a thermal conditioning module 800. This can advantageously result in more a robust, more efficient and/or otherwise improved or enhanced heating or cooling module 800, a smaller module 800 and/or one or more other benefits to a skin treatment device or system.

As discussed in detail herein, fluid being transferred from a fluid source (e.g., a vial or other secured to the handpiece, a bottle or other fluid container of a manifold assembly of a console or tower system that is placed in fluid communication with a handpiece, etc.) can be thermally conditioned at the fluid source, either in lieu of or in addition to thermally conditioning the fluid once it leaves the fluid source (e.g., to be delivered to the distal end of the handpiece or other component that contacts skin tissue).

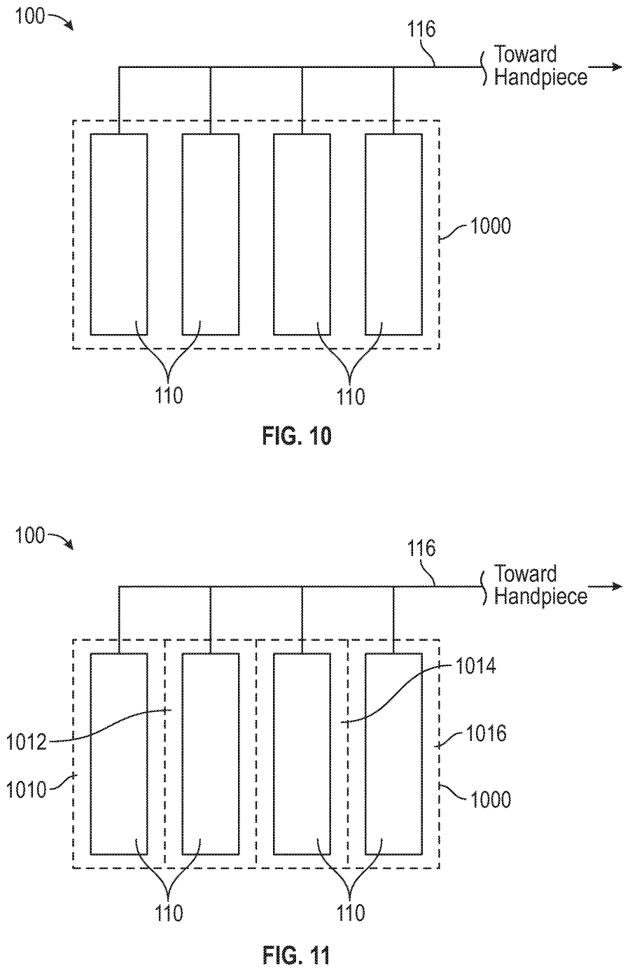

FIG. 10 schematically illustrates one embodiment of a manifold assembly 100 that is configured to receive one or more bottles or other fluid containers 110. As discussed above, such a manifold assembly or other fluid distribution network or system 100 can permit a user to deliver one, two or more treatment fluids and/or other treatment materials contained in one, two or more bottles or other containers 110 secured to the manifold assembly 100. In some embodiments, the manifold assembly 100 and/or another portion of the console, tower and/or other system or component in or with which the manifold assembly 100 is associated, can be configured to thermally condition (e.g., heat and/or cool) the treatment fluid contained in one or more of the bottles or other containers 110 secured to the manifold assembly 100.

In some embodiments, as schematically depicted in FIG. 10, all the bottles or other treatment fluid containers 110 secured to a manifold assembly or other fluid distribution network 100 are configured to be thermally conditioned in the same manner. For instance, a single thermal conditioning device or system 1000 can be used to selectively heat and/or cool the treatment fluids contained in the various bottles or containers 110 secured to the manifold assembly 100.

However, as schematically illustrated in FIG. 11, the thermal conditioning device or system 1000 can comprise two or more distinct thermal conditioning zones or regions 1010, 1012, 1014, 1016. For example, in some embodiments, the thermal conditioning device or system 1000 can include a separate or distinct thermal conditioning zone or region 1010, 1012, 1014, 1016 for each bottle or container 110 secured to the manifold assembly 100. In other embodiments, two or more bottles or containers 110 can be configured to be thermally conditioned by a single thermal conditioning zone or region 1010, 1012, 1014, 1016 of a thermal conditioning device or system 1000, as desired or required.

Accordingly, in some embodiments, the temperature of a treatment fluid or material exiting a manifold assembly 100 can be identical or substantially identical for all treatment fluids or materials passing from one or more of the various bottles or containers 110 to the conduit 116 that is in fluid communication with a handpiece, per the schematic representation of FIG. 10. Alternatively, the temperature of treatment fluids or materials contained in bottles or other containers 110 secured to a manifold assembly or other fluid distribution network 100 can be individually controlled and varied, per the schematic representation of FIG. 11.

Irrespective of whether separate thermal control is provided to the various treatment fluids or materials contained in the bottles or containers 110 secured to a manifold assembly 100, any one of a variety of types of thermal conditioning devices or systems 1000 can be used to thermally condition such treatment fluids or materials. For example, in some embodiments, the thermal conditioning device or system 1000 comprises a liquid or other fluid-based system to maintain treatment fluids and other materials contained in bottles or other containers at a target or desired temperature or temperature range. For example, a simple heating and/or cooling bath of water (e.g., ice water, ice bath, fluid bath, other cooled liquids, cold water or liquid, thermally-conditioned water or other liquid, etc.) can be used to heat and/or cool treatment fluids and/or other materials contained in the bottles or other containers 110 secured to a manifold assembly 100.

In such arrangements, the bottles or other containers 110 secured to a manifold assembly 100 can be provided with sufficient residence time prior to use to achieve the desired temperature value or range. For example, in some embodiments, such a residence time can be 0.2 to 1 hours, 1 to 2 hours, 2 to 4 hours, greater than 4 hours (e.g., 4 to 5, 5 to 6, 6 to 7, 7 to 8, 8 to 9, 9 to 10 hours, greater than 10 hours, etc.), as desired or required. In other embodiments, such a time period can be less than 0.2 hours or greater than 4 hours, such as, for example and without limitation, 0 to 1, 1 to 2, 2 to 3, 3 to 4, 4 to 5, 5 to 6, 0 to 6, 6 to 7, 7 to 8, 8 to 9, 9 to 10, 10 to 11, 11 to 12, 6 to 12 hours, 0 to 0.2 hours, 0 to 0.1 hours, 0.1 to 0.2 hours, values between the foregoing ranges, etc.). In some embodiments, a separate water, liquid and/or other fluid cooling system is provided to cool and/or heat the water or other liquid that is being provided to the thermal conditioning device or system 1000 (and/or the separate/distinct zones or regions 1010, 1012, 1014, 1016).

In other embodiments, a separate cooling and/or heating method is used to provide the desired or required level of thermal conditioning to the thermal conditioning device or system 1000 and/or the separate/distinct zones or regions thereof 1010, 1012, 1014, 1016. For example, separate heating or cooling zones can be created by providing heated or cooled air or other gases to the thermal conditioning device or system 1000 and/or the separate/distinct zones or regions 1010, 1012, 1014, 1016.

For any of the thermal conditioning device or system embodiments 1000 disclosed herein, thermal conditioning can be achieved via convective and/or conductive heat transfer. Further, the system can include one or more feedback loop features, components and/or devices to maintain the heat transfer (e.g., heating, cooling) to a desired level or range, as desired or required. Such features, components and/or devices can include, for example and without limitation, one or more of the following: sensors (e.g., temperature sensors), processors, wiring, other communication methods or protocols, etc.

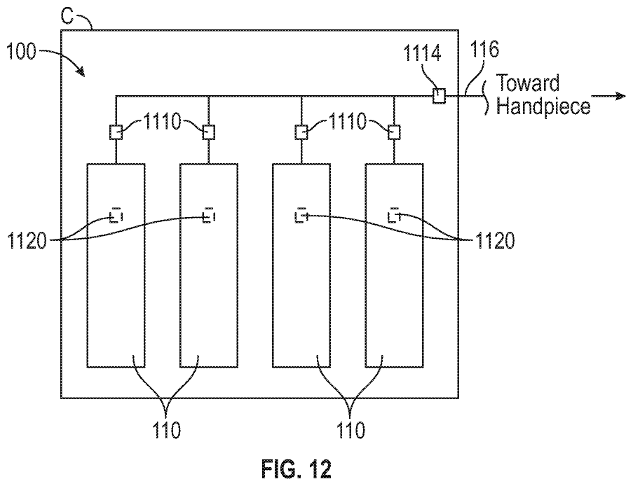

As illustrated in FIG. 12, a manifold system 100 can include one or more thermal conditioning devices or components 1110, 1120 that are configured to heat and/or cool treatment fluids and/or other materials contained in bottles or other containers 110. For example, such thermal conditioning devices or components 1110 can be positioned downstream (e.g., relative to the direction of flow or material transfer) of the corresponding bottle or other container 110. In some embodiments, the thermal conditioning device or component 1110 comprises a thermal conditioning module or device similar to those discussed herein with reference to FIGS. 5 to 7C, and can include, without limitation, one or more of the following: a Peltier or thermoelectric device, another heat pump device, a resistive heating device, another type of heating or cooling device (e.g., conductively, convectively, etc.) and/or the like.

With continued reference to FIG. 12, the thermal conditioning devices or components 1110 can be positioned such that they can selectively heat or cool fluid and/or other treatment materials exiting each bottle or container 110 secured to the manifold assembly 100. In some embodiments, a skin treatment system can include one or more thermal conditioning devices or components 1114 that are configured to thermally condition (e.g., heat and/or cool) treatment fluids and/or materials after they have been combined into a single fluid conduit 116 (e.g., at a location downstream of all the separate fluid streams from the various bottles or containers 110 secured to the manifold system 100). Such thermal conditioning devices or components 1114 can be used in lieu of or in addition to any thermal conditioning devices or components 1110 associated with a specific bottle or container 110 (or a specific grouping of bottles or containers 110).

According to some embodiments, any thermal conditioning devices or components 1110, 1114 included in a particular skin treatment system or component thereof can be positioned along and/or within a tower or console system C. However, in other embodiments, one or more thermal conditioning devices or components 1110, 1114 are separate of any one or more other portions or components of a skin treatment device or system, as desired or required.

For any of the embodiments disclosed herein, thermal conditioning of fluids (e.g., heating and/or cooling) can be provided both at the fluid source (e.g., bottle or other container 110 secured to a manifold assembly 100, a vial or other container secured to a handpiece, etc., as schematically illustrated in FIGS. 10 to 12) and/or one or more locations within or near the handpiece assembly (see, for example, the disclosure herein related to FIG. 5). In other embodiments, however, thermal conditioning is configured to occur only at the fluid source or at least one location within or near the handpiece assembly. In some embodiments, fluid source thermal conditioning is used to strategically raise and/or lower fluid temperature at the source to make it possible or perhaps easier (e.g., faster, more cost-efficiently, etc.) to attain final fluid temperature via secondary thermal conditioning using a handpiece.

In some embodiments, a skin treatment system, regardless of what systems, subsystems, components, etc. it utilizes to heat or cool treatment fluids being delivered to a skin surface, can be configured to heat or cool fluids by 0 to 20 degrees Celsius (e.g., 0 to 5, 5 to 10, 10 to 20, 0 to 10, 0 to 20 degrees Celsius, temperatures between the foregoing ranges, etc.), as desired or required. In some embodiments, a skin treatment system, regardless of what systems, subsystems, components, etc. it utilizes to heat or cool treatment fluids being delivered to a skin surface, can be configured to heat or cool fluids to a temperate of 0 to 70 degrees Celsius (e.g., 0 to 5, 5 to 10, 10 to 20, 20 to 30, 30 to 40, 40 to 50, 0 to 10, 0 to 20, 0 to 30, 0 to 40, 0 to 50, 0 to 60, 0 to 70, 5 to 10, 5 to 20, 5 to 30, 5 to 50, 5 to 50, 10 to 20, 10 to 30, 10 to 40 10 to 50, 20 to 30, 20 to 40, 20 to 50, 30 to 40, 30 to 50, 40 to 50, 30 to 50, 50 to 60, 60 to 70, 50 to 70 degrees Celsius, temperatures between the foregoing ranges, etc.), as desired or required.

For any of the embodiments disclosed herein, a handpiece or device can include the necessary control functions to permit an aesthetician, other professional or other user of the handpiece or device to obtain information (e.g., temperature measurements) and/or make adjustments to the operation of the handpiece or device during use (e.g., increase/decrease flowrate, adjust temperature of fluid/skin surface, etc.). Such control can be accomplished with one or more controllers (e.g., buttons, switches, dials, touchscreens, etc.), audible commands, gesture control and/or the like. Any controllers or input devices or technologies can be incorporated into the handpiece or device itself, a separate system or device (e.g., a console, tower or another component of a skin treatment system, a computer, tablet, smartphone or other separate computing device, etc.).

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, sub-ranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

1. A handpiece for use in skin treatment procedure, the handpiece comprising:
a main body;
a tip positioned along a distal end of the main body; and
a thermal conditioning assembly or module, the thermal conditioning assembly or module being configured to selectively heat and/or cool treatment fluid being transferred to the tip;
wherein the thermal conditioning assembly or module comprises a fluid inlet and a fluid outlet, wherein a fluid pathway extending between the fluid inlet and the fluid outlet comprises at least one turn;
wherein the thermal conditioning assembly or module comprises a heating or cooling component and at least one heat transfer component adjacent the heating or cooling component at or near the fluid pathway; and
wherein the at least one heat transfer component is configured to selectively heat or cool treatment fluid passing through the fluid pathway when the heating or cooling component is electrically energized or activated.

2. The handpiece of claim 1,
wherein treatment fluid is heated or cooled conductively;
wherein the thermal conditioning assembly or module is configured to both heat and cool treatment fluid; and
wherein the at least one heat transfer component at least partially extends into the fluid pathway.

3. (canceled)

4. (canceled)

5. The handpiece of claim 1, wherein the thermal conditioning assembly or module is configured to both heat and cool treatment fluid.

6. (canceled)

7. (canceled)

8. A method for treating skin, the method comprising:
performing a skin treatment procedure using a tip of a handpiece, the handpiece comprising a main body and the tip, wherein the tip is positioned at or near a distal end of the main body; and
selectively heating and/or cooling a treatment fluid being transferred to the tip using a thermal conditioning assembly or module of the handpiece;
wherein selectively heating and/or cooling a treatment fluid comprises using a thermal conditioning assembly or module;
wherein the thermal conditioning assembly or module comprises a fluid inlet and a fluid outlet, wherein a fluid pathway extending between the fluid inlet and the fluid outlet comprises at least one turn;
wherein the thermal conditioning assembly or module comprises a heating or cooling component and at least one heat transfer component extending between the heating or cooling component at the fluid pathway; and
wherein the at least one heat transfer component is configured to selectively heat or cool treatment fluid passing through the fluid pathway when the heating or cooling component is electrically energized.

9. The method of claim 8,
wherein treatment fluid is heated or cooled conductively; and
wherein the thermal conditioning assembly or module is configured to both heat and cool treatment fluid.

10. The method of claim 8, wherein treatment fluid is heated or cooled conductively.

11. The method of claim 8, wherein treatment fluid is heated or cooled convectively.

12. The method of claim 8, wherein the thermal conditioning assembly or module is configured to both heat and cool treatment fluid.

13.-25. (canceled)

26. A skin treatment system comprising a handpiece configured to contact a skin surface being treated, the skin treatment system comprising:
at least one fluid source configured to contain a treatment fluid;
a handpiece;
a fluid conduit placing the handpiece in fluid communication with the at least one fluid source; and
a thermal conditioning assembly, the thermal conditioning assembly being configured to selectively heat and/or cool treatment fluid being transferred from the at least one fluid source to the handpiece;
wherein the thermal conditioning assembly is located between an outlet of the at least one fluid source and the fluid conduit.

27. The system of claim 26, wherein the thermal conditioning assembly is configured to heat and/or cool treatment fluid convectively.

28. The system of claim 26, wherein the thermal conditioning assembly is configured to heat and/or cool treatment fluid conductively.

29. The system of claim 26, further comprising at least one thermal conditioning module positioned within the handpiece, said at least one thermal conditioning module configured to heat and/or cool fluid entering the handpiece from the fluid conduit.

30. The system of claim 26, wherein the thermal conditioning assembly comprises an ice bath.

31. The system of claim 26, wherein the thermal conditioning assembly comprises a fluid bath to heat or cool the treatment fluid.

32. The system of claim 29, wherein treatment fluid is heated or cooled conductively using the at least one thermal conditioning module in the handpiece.

33. The system of claim 29, wherein treatment fluid is heated or cooled convectively using the at least one thermal conditioning module in the handpiece.

34. The system of claim 29, wherein the at least one thermal conditioning module comprises an electric heater.

35. The system of claim 29, wherein the at least one thermal conditioning module comprises a thermoelectric device or another type of heat pump.

36. The system of claim 26, wherein the thermal conditioning assembly is configured to both heat and cool treatment fluid being transferred from the at least one fluid source to the handpiece.
